SEQUENCE LISTING (6 sequences)

SEQ ID NO: 1 - DNA, 20 bp, artificial
  beta-III-tubulin forward primer
atccggaccgcatcatgaac

SEQ ID NO: 2 - DNA, 20 bp, artificial
  beta-III-tubulin reverse primer
accatgttgacggccagctt

SEQ ID NO: 3 - DNA, 23 bp, artificial
  NEFM forward primer
aaatcgctgcgtacagaaaactc

SEQ ID NO: 4 - DNA, 21 bp, artificial
  NEFM reverse primer
tgcttcctgcaaatgtgctaa

SEQ ID NO: 5 - DNA, 19 bp, artificial
  G3PDH control - forward primer
gaaggtgaaggtcggagtc

SEQ ID NO: 6 - DNA, 20 bp, artificial
  G3PDH control - reverse primer
gaagatggtgatgggatttc

(19) United States
(12) Patent Application Publication
(10) Pub. No.: US 2013/0065304 A1
(43) Pub. Date: Mar. 14, 2013

(54) DIFFERENTIATION PROCESS OF MESENCHYMAL STEM CELLS AND THERAPEUTIC USE THEREOF

(71) Applicant: Erica MOLINO, Magliano Alfieri (Cuneo) (IT)

(72) Inventor: Erica MOLINO, Magliano Alfieri (Cuneo) (IT)

(73) Assignee: Davide Vannoni, Torino (IT)

(21) Appl. No.: 13/603,243

(22) Filed: Sep. 4, 2012

Related U.S. Application Data
(63) Continuation of application No. 12/964,941, filed on Dec. 10, 2010.

(51) Int. Cl. C12N 5/0797; C12N 5/0793

(52) U.S. Cl. 435/377; 435/325

(57) ABSTRACT

Process for inducing differentiation of mesenchymal stamina cells into neuroblasts and/or neurons that envisions the use of a differentiation solution consisting of retinoic acid and ethanol.

### FIELD OF THE INVENTION

The present invention concerns a process for differentiation of mesenchymal stem cells into cells with a specific phenotype for their successive therapeutic use.

TECHNICAL BACKGROUND Mesenchymal stem cells are present in the medullary stroma [1]. They are pluripotent cells that, if appropriately directed, have the capacity to replicate and differentiate both in vivo and in vitro into a variety of cell types such as osteoblasts, chondrocytes [2], myocytes [3], neuronal cells [4], to cite only a few examples. This differentiation potential makes mesenchymal stem cells an important therapeutic resource for diverse pathologies [5].

The adult nervous system has a limited capacity for endogenous regeneration both in terms of cellular replication and successive reorganisation of functionally adequate circuits.

Therefore, the use of mesenchymal stem cells represents an instrument for the treatment of neurodegenerative pathologies, such as for example Parkinson's disease, Alzheimer's disease, progressive supranuclear palsy, multiple system atrophy, amyotrophic lateral sclerosis, Huntington's chorea [6-9] or following trauma such as stroke and cerebral or spinal traumas.

In general practice mesenchymal stem cells are obtained from bone marrow aspirates or by means of blood sampling.

For most therapeutic applications it is useful to induce the differentiation of the mesenchymal stem cells into the cellular phenotype of interest.

For treating nervous system pathologies, such as neurodegenerative pathologies or peripheral neuropathologies, the mesenchymal stem cells must be induced to differentiate into neuroblasts and/or neurons.

There are indications in the scientific literature regarding substances capable of inducing the differentiation of mesenchymal stem cells into neuroblasts and/or neurons. However, these indications are not applicable in clinical practice, since the relative dosages of such substances or the relative application criteria may cause collateral effects in patients, for example teratogenesis, leading to new pathologies.

### SUMMARY OF THE INVENTION

Therefore, considering these preambles, the need is felt for improving solutions that allow differentiation of mesenchymal stem cells into cells with a specific phenotype.

The object of the present description is to provide such improving solutions.

According to the invention, said objective is obtained by means of the solution specifically recalled in the attached claims, which constitute an integral part of the present description.

The present invention concerns a process for inducing the differentiation of mesenchymal stem cells into a cell type of interest, specifically neuroblasts and/or neurons, that envisions the use of a cellular differentiation solution constituted of an alcoholic solution of retinoic acid. The choice of this specific solution based only on retinoic acid and the mode of use of such solution provide a substantial advantage to the procedure because it minimises the toxic (teratogenic) effects of exposing cells to retinoic acid.

### DETAILED DESCRIPTION OF SOME EMBODIMENTS

The invention will now be described in detail, by way of example only, with reference to the attached figures, in which:

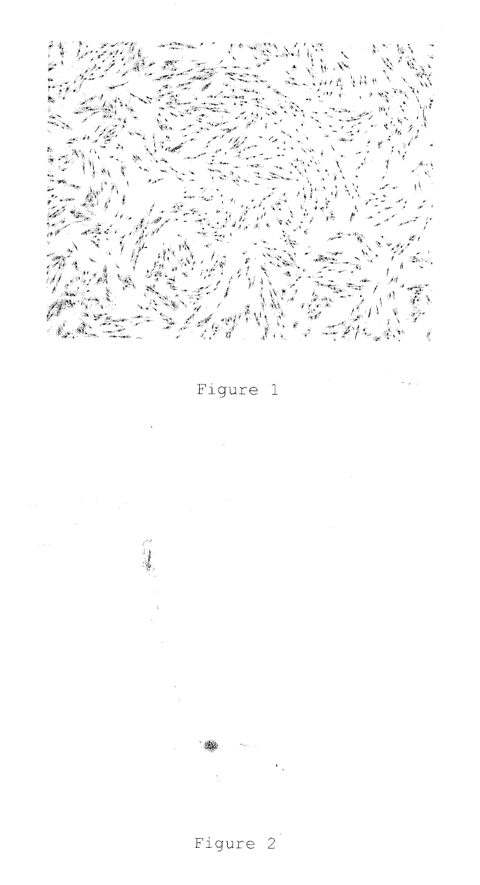

FIG. 1: Optical micrograph (20× magnification) of undifferentiated mesenchymal stem cells in culture.

FIG. 2: Optical micrograph (40× magnification) of two neuroblasts at different levels of differentiation.

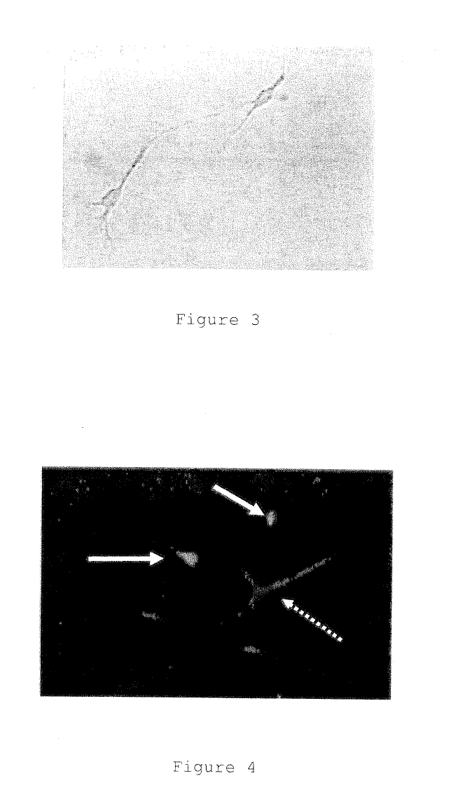

FIG. 3: Optical micrograph (40× magnification) illustrating the neuronal morphology obtained from mesenchymal stem cells differentiated with the method described in the present description; the presence of axons, dendrites and dendritic spines are evident.

FIG. 4: Fluorescence micrograph obtained from immunohistochemistry experiments demonstrating the expression of nestin (solid arrow) and vimentin (broken arrow), by mesenchymal stem cells differentiated into neuroblasts with the method described in the present description.

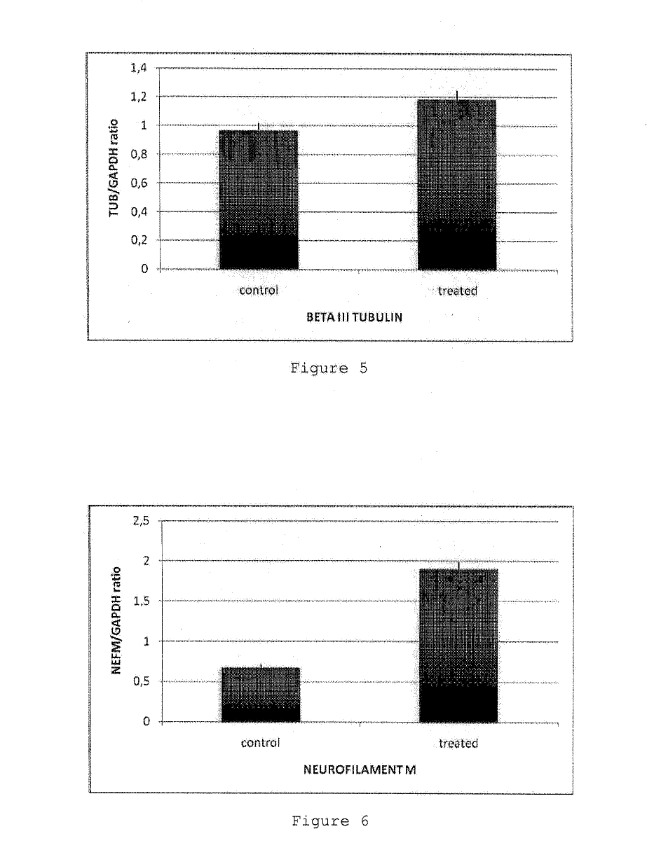

FIG. 5: Real-time RT-PCR results of the neuronal marker expression in control, not treated mesenchymal stem cells (“control”) and differentiated cells (“treated”). Values showed in the histogram derive from the ratio between Beta tubulin and the housekeeping gene GAPDH expression.

FIG. 6: Real-time RT-PCR results of the neuronal marker expression in control, not treated mesenchymal stem cells (“control”) and differentiated cells (“treated”). Values showed in the histogram derive from the ratio between Neurofilament M and the housekeeping gene GAPDH expression.

In the following description, numerous specific details are given to provide a thorough understanding of the embodiments. The embodiments can be practiced without one or more of the specific details, or with other methods, components, materials, etc. In other instances, well-known structures, materials or operations are not shown or described in detail to avoid obscuring certain aspects of the embodiments.

Reference throughout the present specification to “one embodiment” or “an embodiment” means that a particular feature, structure or characteristic described in connection with the embodiment is included in at least one embodiment. Thus, the phrase “in one embodiment” or “in an embodiment” in various places throughout the present specification are not necessarily all referring to the same embodiment. Furthermore, the details of features, structures, or characteristics may be combined in any suitable manner in'one or more embodiments.

The headings provided herein are for convenience only and do not interpret the scope or meaning of the embodiments.

The present invention envisions an innovative procedure for inducing the differentiation of mesenchymal stem cells into neurons/neuroblasts, where the mesenchymal stem cells—obtained from patients according to techniques known in the art—can be induced to differentiate both when suspended in physiological solution and when adherent to the walls of a container into which they were introduced after harvesting from the patient.

The differentiation solution described in such invention is characterised by two substances: retinoic acid (the neuronal differentiation factor) and 98% ethanol.

The results presented in the literature describe a differentiation solution constituted of retinoic acid dissolved in culture media and administered at a concentration lower than described, for example in Schegelskaya et al., Russian Journal of Developmental Biology, 2003, 34:185-191.

The advantage of the solution described in the invention is provided by its simple composition, requiring only two substances.

Evidence is presented in the literature that mesenchymal cells progressively acquire a neuronal phenotype over the course of several days.

On the contrary, the solution used in the invention induces a very rapid differentiation of the cells: after about 20 minutes of treatment it is possible to obtain neuroblasts and after about 2 hours of treatment completely formed and functional mature neurons are already present.

The advantage is that the brief duration of the contact of the biological material with the inducing substance limits the toxic effects of retinoic acid on the differentiated cells. Therefore, such procedure can be used for the application of mesenchymal stem cells in the treatment of neurodegenerative diseases and of various forms of traumatic central or peripheral neuropathies.

Before differentiation, the mesenchymal stem cells are expanded—using cellular expansion techniques known in the art—to obtain a clinically useful number and overcome one of the primary limitations in their therapeutic use: often when these cells are harvested from marrow they are not numerous enough. Outside of the body (ex vivo), they maintain a good proliferative capacity and are able to adhere to surfaces such as glass and plastic, which are commonly used for culturing cells in the laboratory.

The two different operations will now be described in detail.

a) Differentiation into Neuroblasts/Neurons in Suspension

To the mesenchymal stem cells suspended in physiological solution 6 μl/ml of neuronal differentiation solution are added.

The solution obtained is delicately resuspended and maintained at 37° C. for a period comprised between 20 minutes and 2 hours, preferably between 40 minutes and 1 hour and 30 minutes, in function of the maturation state (neuroblasts-neurons) desired.

At two hours mature neurons with dendrites and axons completely formed and functional are present (see FIGS. 1 and 2 above).

The solution for neuronal differentiation is composed thusly:
10 ml of 98% ethanol
10 mg of retinoic acid.

The relative quantities indicated above are to be considered exclusively as a practical indication for obtaining a differentiation solution having a retinoic acid concentration of 3×10⁻³ M.

The neuronal differentiation solution is agitated to dissolve the retinoic acid and maintained refrigerated at 4° C.

b) Differentiation into Neuroblasts/Neurons in Adhesion

Six microlitres per millilitre (6 μl/ml) of the neuronal differentiation solution prepared just before use (maximum 1 hour) and stored at +4° C. in the dark is added to the mesenchymal stem cells adherent to the wall of a culture flask.

The flask is placed in an incubator for a period comprised between 20 minutes and 2 hours, preferably between 40 minutes and 1 hour and 30 minutes, in function of the maturation state (neuroblasts-neurons) desired.

At two hours mature neurons are present with completely formed and functional dendrites and axons, the morphology of which can be appreciated (see Figures and 4) and on which it is possible to conduct immunohistochemical and electrophysical tests.

The solution for neuronal differentiation is composed thusly:
10 ml of 98% ethanol
10 mg of retinoic acid.

The relative quantities indicated above are to be considered exclusively a practical indication for obtaining a differentiation solution having a retinoic acid concentration of 3×10⁻³ M.

With respect to what is present in the literature, the formula for differentiation described herein is simplifying (in fact, requiring only two substances) and does not envision a long duration of the biological material in culture together with the differentiation inducing substance. This minimises the possible toxic effects of retinoic acid on the cells, which in this way can be employed for human use in cellular therapies for neurodegenerative and traumatic (spinal and cerebral) pathologies, as well as for various forms of peripheral neuropathies.

c) Characterisation of Differentiated Mesenchymal Stem Cells

Quantitative Real time RT-PCR on cells treated with the differentiating solution for one 1 hour and 30 minutes was performed in order to verify the expression of neuronal markers.

In particular, β III-tubulin and Neurofilament M (NF-M) expression were examined in clonally expanded BMSC, used as control, and in BMSC induced to express neuronal phenotype. Real Time experiments were performed using three different primary cell lines.

Neurofilaments are the 10 nanometer (10nm) intermediate filaments found specifically in the core of neuronal axons.

Neurofilaments are heteropolymers composed of three type IV polypeptides: NF-L, NF-M, and NF-H (for low, middle, and high molecular weight). They are responsible for the radial growth of an axon and determine axonal diameter.

During axonal growth, new neurofilament subunits are incorporated all along the axon in a dynamic process that involves the addition of subunits along the filament length, as well as the addition of subunits at the filament ends. After an axon has grown and connected with its target cell, the diameter of the axon may increase as much as fivefold. Neurofilaments are repulsive. This is because their purpose is to set the diameter of dendrites and axons. They do this by repelling each other because of their polarity and move away from each other.

The level of neurofilament gene expression seems to directly control axonal diameter, which in turn controls how fast electrical signals travel down the axon.

Tubulin is one of several members of a small family of globular proteins. The most common members of the tubulin family are α-tubulin and β-tubulin, the proteins that make up microtubules. Each has a molecular weight of approximately 55 kiloDaltons. Microtubules are assembled from dimers of α- and β-tubulin. To form microtubules, the dimers of α- and β-tubulin bind to GTP and assemble onto the (+) ends of microtubules while in the GTP-bound state. After the dimer is incorporated into the microtubule, the molecule of GTP bound to the β-tubulin subunit eventually hydrolyzes into GDP through inter-dimer contacts along the microtubule protofilament [4]. Whether the β-tubulin member of the tubulin dimer is bound to GTP or GDP influences the stability of the dimer in the microtubule.

Class III β-tubulin is a microtubule element expressed exclusively in neurons, and is a popular identifier specific for neurons in nervous tissue.

β III-tubulin and NF-M expression in control cells and in differentiated MSC was examined as follows:

total RNAs were extracted from samples using TRIzol reagent (Invitrogen) according to the manufacturer's protocol and subjected to reverse transcription by Omniscript RT Kit (Qiagen, Valencia, Calif., USA), following the manufacturer's instructions. The total RNA from samples was measured by real-time quantitative RT-PCR using PE ABI Prism@ 7700 Sequence Detection System (Perkin Elmer, Wellesley, Mass., USA) and the SYBR Green method, following manufacturer's instructions. Gene specific primers for RT-PCR analysis was generated using the NCBI Primer-BLAST software (http://www.ncbi.nlm.nih.gov/tools/primer-blast/) and NCBI Reference Sequences (whole genome assembly released by the Macaca mulatta Genome Sequencing Consortium as Mmul_{—}051212, February 2006, whole genome shotgun sequence, http://www.hgsc.bcm.tmc, edu/projects/rmacaque/) provided by NIH online resources. The sequences of forward and reverse primers were:

β III-tubulin:
(forward - SEQ ID No.: 1)
5′-ATCCGGACCGCATCATGAAC-3′,
(reverse - SEQ ID No.: 2)
5′-ACCATGTTGACGGCCAGCTT-3′;
NF-M:
(forward - SEQ ID No.: 3)
5′-AAATCGCTGCGTACAGAAAACTC-3′,
(reverse - SEQ ID No.: 4)
5′-TGCTTCCTGCAAATGTGCTAA-3′.

For endogenous control, the expression of glyceraldehyde 3-phosphate dehydrogenase (G3PDH) gene was examined.

The sequences for human G3PDH primers were: 5′-GAAGGTGAAGGTCGGAGTC-3′ (forward—SEQ ID No.:5), 5′-GAAGATGGTGATGGGATTTC-3′ (reverse—SEQ ID No.:6).

Relative transcript levels were determined from the relative standard curve constructed from stock cDNA dilutions and were divided by the target quantity of the calibrator, following manufacturer's instructions.

As shown in FIG. 5, β III-tubulin was positive in the control, although the expression was considerably lower than in treated cells, while neuronal differentiation treatment enhanced III-tubulin expression in a statistically significant way.

As shown in FIG. 6, the control group of BMSC exhibited weak expression of mature neuronal markers NF-M. In contrast, mature neuronal markers were abundant in the groups treated with neuronal differentiation medium.

Naturally, without prejudice to the underlying principle of the invention, the structural details and the embodiments may vary, even appreciably, with reference to what has been described by way of example only, without departing from the scope of the invention as defined by the annexed claims.

### BIBLIOGRAPHY

[1] Human bone marrow mesenchymal stem cells in vivo. E. Jones and D. McGonagle. Rheumatology 2008; 47; 126-131
[2] Bone marrow stromal cells: nature, biology, and potential applications. Bianco, P., Riminucci, M., Gronthos, S. and Robey, P G. Stem cells 2001; vol 19 no 3; 180-192
[3] Multilineage potential of adult human mes-enchymal stem cells. Pittenger, M. F., A. M. Mackay, S. C. Beck, et al. Science 284: 143-147.
[4] Neurons Derived From Human Mesenchymal Stem Cells Show Synaptic Transmission and Can Be Induced to Produce the Neurotransmitter Substance P by Interleukin-1α. Kyung Jin Cho, Katarzyna A. Trzaska, Steven J. Greco, Joseph McArdle, Fu Shun Wang, Jiang-Hong Ye, Pranela Rameshwar Stem Cells. 2005;23:383-391.
[5] Mesenchymal stem cells: biological characteristics and potential clinical applications. Kassem M. Cloning Stem Cells. 2004; 6(4):369-74.
[6] Cell therapy for Parkinson's disease: II. Somatic stem cell-based applications. Anisimov S V. Adv Gerontol. 2009; 22(1):150-66.
[7] Bone marrow-derived mesenchymal stem cells reduce brain amyloid-beta deposition and accelerate the activation of microglia in an acutely induced Alzheimer's disease mouse model. Lee J K, Jin H K, Bae J S. Neurosci Lett. 2009 Jan. 30; 450(2):136-41. Epub 2008 Dec. 6.
[8] Adult neurotrophic factor-secreting stem cells: a potential novel therapy for neurodegenerative diseases. Sadan 0, Shemesh N, Cohen Y, Melamed E, Offen D. Isr Med Assoc J. 2009 April; 11(4):201-4.
[9] Microanatomical evidences for potential of mesenchymal stem cells in amelioration of striatal degeneration. Amin E M, Reza B A, Morteza B R, Maryam M M, All M, Zeinab N. Neurol Res. 2008 December; 30(10):1086-90.

1. Process for inducing differentiation of mesenchymal stem cells into neuroblasts and/or neurons, characterised in that said process comprises the use of a differentiation solution consisting of retinoic acid and ethanol.

2. Process for inducing differentiation according to claim 1, wherein said differentiation solution comprises retinoic acid in a concentration comprised between 1×10⁻³ M and 6×10⁻³ M, preferably equal to 3×10⁻³ M.

3. Process for inducing differentiation according to claim 1 or claim 2, wherein said mesenchymal stem cells are maintained in said differentiation solution for a period comprised between 20 minutes and 2 hours, preferably between 40 minutes and 1 hour and 30 minutes.

4. Neuroblasts and/or neurons obtained using the process according to any of claims 1 to 3 for use in the treatment of neurodegenerative pathologies and/or central or peripheral traumatic neuropathies.
